(19) United States
(12) Patent Application Publication
(10) Pub. No.: US 2014/0371506 A1
(43) Pub. Date: Dec. 18, 2014

(54) PROCESS FOR REMOVING ONE OR MORE SULFUR COMPOUNDS, AND A VESSEL RELATING THERETO

(71) Applicant: UOP, LLC, Des Plaines, IL (US)

(72) Inventor: Luigi Laricchia, Arlington Heights, IL (US)

(21) Appl. No.: 13/920,477

(22) Filed: Jun. 18, 2013

(51) Int. Cl. C07C 7/00

(52) U.S. Cl. CPC C07C 7/005
USPC 585/802; 422/262

(57) ABSTRACT

One exemplary embodiment can be a process for removing one or more sulfur compounds from one or more hydrocarbons. The process may include passing a hydrocarbon stream from a prewash zone containing a coalescing zone to an extraction zone. Often, the zones are contained within a single vessel and the coalescing zone comprises an oleophilic media.

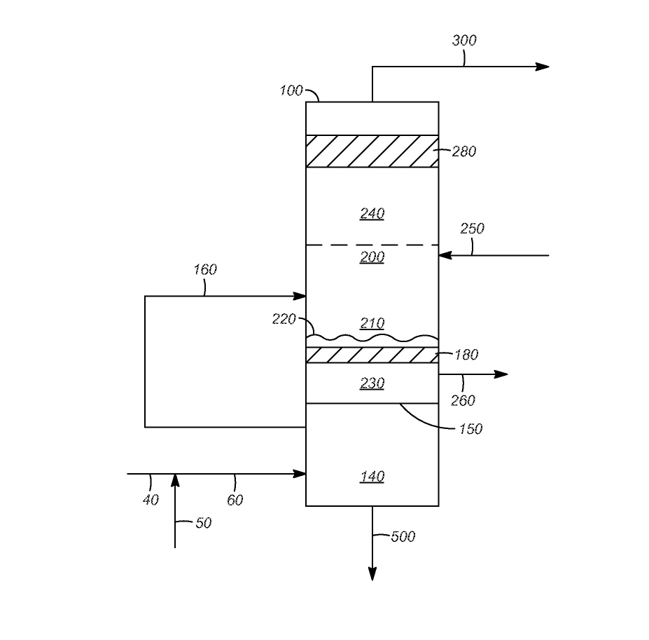

### FIELD OF THE INVENTION

This invention generally relates to a process for removing one or more sulfur compounds from one or more hydrocarbons, and a vessel relating thereto.

### DESCRIPTION OF THE RELATED ART

Caustic carryover in the hydrocarbon streams, such as fuel gas and liquefied petroleum gas, is one of the major causes of off-spec products, high caustic consumption, corrosion of carbon steel, and major upsets caused in processes downstream of caustic sweetening and/or extraction. Desirably, reducing caustic carryover can minimize downstream upsets. One mechanism for minimizing carryover may be avoiding hydrocarbon contamination. However, hydrocarbons, particularly liquefied petroleum gas derived from fluid catalytic cracking or coker units, can cause strong emulsions in the circulating caustic, potential gums across an oxidizing vessel, poor separation of disulfide oil from caustic in a separation vessel, and ultimately carryover of caustic into downstream units. It is desirable to avoid contaminating rich caustic with hydrocarbons at the bottom of an extractor column. Additionally, it is also preferable to coalesce lean caustic from the hydrocarbon products without the use of expensive downstream equipment. Hence, there is a desire to improve the efficiency of extraction and/or sweetening processes.

### SUMMARY OF THE INVENTION

One exemplary embodiment can be a process for removing one or more sulfur compounds from one or more hydrocarbons. The process may include passing a hydrocarbon stream from a prewash zone containing a coalescing zone to an extraction zone. Often, the zones are contained within a single vessel and the coalescing zone comprises an oleophilic media.

Another exemplary embodiment may be a process for removing one or more sulfur compounds from one or more hydrocarbons. The process may include passing a combined stream having one or more hydrocarbons and an alkali to a prewash zone, obtaining from the prewash zone a hydrocarbon stream and passing the hydrocarbon stream into an extraction zone including a first coalescing zone, mixing the hydrocarbon stream with an alkali stream to obtain a hydrocarbon phase and an alkali phase, and passing at least a portion of the hydrocarbon phase to a settling zone containing a second coalescing zone to obtain a processed hydrocarbon stream.

Another exemplary embodiment may be a process for removing one or more sulfur compounds from one or more hydrocarbons. The process may include passing a combined stream having one or more hydrocarbons and an alkali to a prewash zone, obtaining from the prewash zone a hydrocarbon stream and passing the hydrocarbon stream into an extraction zone including a first coalescing zone, mixing the hydrocarbon stream with an alkali stream to obtain a hydrocarbon phase and an alkali phase, and passing at least a portion of the hydrocarbon phase to a settling zone containing a second coalescing zone to obtain a processed hydrocarbon stream.

A further exemplary embodiment can be a vessel for removing one or more sulfur compounds from one or more hydrocarbons. The vessel can include a prewash zone, an extraction zone downstream of the prewash zone containing a first coalescing zone, and a settling zone downstream of the extraction zone containing a second coalescing zone.

The embodiments disclosed herein can improve the separation between caustic and hydrocarbons in both rich caustic and hydrocarbon product streams using multi-stage coalescing media. The coalescing media may include an optionally coated mesh blanket, corrugated sheet media, or other accepted liquid-liquid coalescing media. The coalescing media installed at the bottom of the extractor column can have oleophilic properties because the caustic is often a continuous phase. The coalescing media may be at the top of an extraction and/or a settling zone and may have hydrophilic properties because the hydrocarbon may be in a continuous phase.

### DEFINITIONS

As used herein, the term “stream” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C1, C2, C3 . . . Cn where “n” represents the number of carbon atoms in the one or more hydrocarbon molecules. Furthermore, a superscript “+” or “−” may be used with an abbreviated one or more hydrocarbons notation, e.g., C3⁺ or C3⁻, which is inclusive of the abbreviated one or more hydrocarbons. As an example, the abbreviation “C3⁺” means one or more hydrocarbon molecules of three carbon atoms and/or more. In addition, the term “stream” may be applicable to other fluids, such as aqueous and non-aqueous solutions of alkaline or basic compounds, such as sodium hydroxide.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally about 50%, and preferably about 70%, by mole, of a compound or class of compounds in a stream. If referring to a solute in solution, e.g., one or more disulfide compounds in an alkaline solution, the term “rich” may be referenced to the equilibrium concentration of the solute. As an example, about 5%, by mole, of a solute in a solvent may be considered rich if the concentration of solute at equilibrium is about 10%, by mole.

As used herein, the term “substantially” can mean an amount of at least generally about 80%, preferably about 90%, and optimally about 99%, by mole, of a compound or class of compounds in a stream.

As used herein, the term “coupled” can mean two items, directly or indirectly, joined, fastened, associated, connected, or formed integrally together either by chemical or mechanical means, by processes including stamping, molding, or welding. What is more, two items can be coupled by the use of a third component such as a mechanical fastener, e.g., a screw, a nail, a bolt, a staple, or a rivet; an adhesive; or a solder.

As used herein, the term “coalescer” may be a media containing an optionally coated metal mesh, glass fibers, or other material to facilitate separation of immiscible liquids of similar density.

As used herein, the term “immiscible” can mean two or more phases that cannot be uniformly mixed or blended.

As used herein, the term “phase” may mean a liquid, a gas, or a suspension including a liquid and/or a gas, such as a foam, aerosol, or fog. A phase may include solid particles. Generally, a fluid can include one or more gas, liquid, and/or suspension phases.

As used herein, the term “alkali” can mean any substance that in solution, typically a water solution, has a pH value greater than about 7.0, and exemplary alkali can include sodium hydroxide, potassium hydroxide, or ammonia. Such an alkali in solution may be referred to as “an alkaline solution” or “an alkaline” and includes caustic, i.e., sodium hydroxide in water.

As used herein, the term “parts per million” may be abbreviated herein as “ppm” and “weight ppm” may be abbreviated herein as “wppm”.

As used herein, the term “mercaptan” typically means thiol and may be used interchangeably therewith, and can include compounds of the formula RSH as well as salts thereof, such as mercaptides of the formula RS⁻M⁺ where R is a hydrocarbon group, such as an alkyl or aryl group, that is saturated or unsaturated and optionally substituted, and M is a metal, such as sodium or potassium.

As used herein, the term “disulfides” can include dimethyldisulfide, diethyldisulfide, and ethylmethyldisulfide, and possibly other species having the molecular formula RSSR′ where R and R′ are each, independently, a hydrocarbon group, such as an alkyl or aryl group, that is saturated or unsaturated and optionally substituted. Typically, a disulfide is generated from the oxidation of a mercaptan-containing caustic and forms a separate hydrocarbon phase that is not soluble in the aqueous caustic phase. Generally, the term “disulfides” as used herein excludes carbon disulfide (CS₂).

As used herein, the weight percent or ppm of sulfur, e.g., “wppm-sulfur” is the amount of sulfur, and not the amount of the sulfur-containing species unless otherwise indicated. As an example, methylmercaptan, CH₃SH, has a molecular weight of 48.1 with 32.06 represented by the sulfur atom, so the molecule is about 66.6%, by weight, sulfur. As a result, the actual sulfur compound concentration can be higher than the wppm-sulfur from the compound. An exception is that the disulfide content in caustic can be reported as the wppm of the disulfide compound.

As used herein, the term “lean” can describe a fluid optionally having been treated and desired levels of sulfur, including one or more mercaptans and one or more disulfides for treating one or more C1-C4 hydrocarbons.

As used herein, the term “regeneration” with respect to a solvent stream can mean removing one or more disulfide sulfur species from the solvent stream to allow its reuse.

As used herein, the terms “degrees Celsius” may be abbreviated “° C.” and the term “kilopascal” may be abbreviated “KPa” and all pressures disclosed herein are absolute.

As depicted, process flow lines in the figures can be referred to, interchangeably, as, e.g., lines, pipes, branches, distributors, streams, effluents, feeds, products, portions, catalysts, withdrawals, recycles, suctions, discharges, and caustics.

### BRIEF DESCRIPTION OF THE DRAWING

The FIGURE is an elevational, cross-sectional view of an exemplary vessel.

### DETAILED DESCRIPTION

Referring to the FIGURE, an exemplary vessel 100 for removing one or more sulfur compounds from one or more hydrocarbons is depicted. The vessel 100 can be utilized in an extraction system for removing one or more thiol compounds from one or more hydrocarbons by, e.g., converting one or more thiol compounds into one or more disulfide compounds. Such systems are disclosed in, e.g., U.S. Pat. No. 7,381,309. The vessel 100 may include a prewash zone 140, a first coalescing zone 180, an extraction zone 200, a settling zone 240, and a second coalescing zone 280.

A hydrocarbon stream 40 upstream of the prewash zone 140 can include one or more C4⁻ hydrocarbons, such as fuel gas or a liquefied petroleum gas, and be provided at a temperature of about 30-about 50° C., and a pressure of about 400-about 1,900 KPa.

Generally, the hydrocarbon stream 40 may be rich in or substantially has one or more C4⁻ hydrocarbons. The hydrocarbon stream 40 may be one or more liquids, gases, or a mixture of one or more gases and liquids. The hydrocarbon stream 40 can be combined with an alkaline or an alkali stream 50 including an alkali, such as at least one of an ammonia, a potassium hydroxide and a sodium hydroxide, in a water solution. Typically, the water solution includes about 10-about 20%, by weight, alkali with the balance water. The streams 40 and 50 can be added together to form a combined stream 60 provided to the vessel 100.

The combined stream 60 is provided to the vessel 100 in the prewash zone 140 for removing hydrogen sulfide by converting to, e.g., sodium sulfide. A side-stream 260 can be withdrawn including primarily an alkaline rich in sulfur compounds, such as one or more thiol compounds. Generally, the side-stream 260 has about 1-about 100 ppm, by weight, of one or more hydrocarbons. The side-stream 260 can be sent to an alkali regeneration zone that can include an oxidation vessel and a disulfide separator. Such alkali regeneration zones are disclosed in, e.g., U.S. Pat. No. 7,381,309. A bottom or purge stream 500, including primarily an alkaline rich in sulfur compounds, may be withdrawn for controlling the level of alkaline in the vessel 100. The purge stream 500 can either be sent for disposal or sent to an alkali regeneration zone as discussed above for the side-stream 260. A lean alkali stream, such as a stream 250, may be returned to the vessel 100 from the alkali regeneration zone.

A stream 160 from the prewash zone 140 can be provided to the extraction zone 200 downstream from the prewash zone 140. A physical barrier 150, such as a plate 150, can separate the zones 140 and 200. The lean alkali stream 250, including about 10-about 20%, by weight, alkali with the balance water may be provided to the extraction zone 200. The stream 160 can separate into a hydrocarbon phase 210 and an alkali phase 230 forming an interface 220. The extraction zone 200 can include a first coalescing zone 180 including an oleophilic media extending across the entire cross-sectional area of the vessel 100. Usually, the oleophilic media includes at least one of a metal mesh that is optionally coated, one or more glass fibers, sand, or anthracite coal. In one exemplary embodiment, the oleophilic media can include an oleophilic coated mesh. Desirably, the coating may be oleophilic and/or hydrophobic usually suited for an aqueous phase. Such a coating may include at least one of a fluoropolymer and polypropylene. Suitable fluoropolymers can include one or more of polytetrafluoroethylene, fluorinated ethylene-propylene, perfluoroalkoxy, and ethylene tetrafluoroethylene. Exemplary fluoropolymers are disclosed in, e.g., U.S. Pat. No. 5,456,661 and U.S. Pat. No. 2,230,654.

A settling zone 240 can be downstream from the extraction zone 200. Usually, there is no physical barrier between the zones 200 and 240. Rather, the extraction zone 200 can transition to the settling zone 240. The settling zone 240 can contain a second coalescing zone 280 including a hydrophilic or oleophobic media for coalescing water droplets extending across the entire cross-sectional area of the vessel 100. Generally, the hydrophilic media includes at least one of a metal mesh that is optionally coated; one or more glass fibers such as fiberglass; or a metal, such as stainless steel, mesh. Desirably, the coating may be oleophobic and/or hydrophilic usually suited for an oil phase. One exemplary hydrophilic coated mesh may include a coating sold under the trade designation COALEX or KOCH-OTTO YORK™ separations technology by Koch-Glitsch, LP of Wichita, Kans.

If the hydrocarbons, such as a fuel gas, are in a gas phase instead of a liquid phase, a demister may be used instead of the second coalescing zone 280. Such a demister may be a vane or mesh and constructed from any suitable material such as a metal, e.g., stainless steel. A processed hydrocarbon stream 300 having no more than about 1 ppm, by weight, sodium ions can be obtained from the settling zone 240 and withdrawn from the vessel 100.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for removing one or more sulfur compounds from one or more hydrocarbons, comprising:
passing a hydrocarbon stream from a prewash zone containing a coalescing zone to an extraction zone, wherein the zones are contained within a single vessel and the coalescing zone comprises an oleophilic media.

2. The process according to claim 1, further comprising passing an alkali stream into the extraction zone wherein the alkali stream comprises at least one of an ammonia, a potassium hydroxide and a sodium hydroxide.

3. The process according to claim 1, wherein the extraction zone contains a hydrocarbon phase and an alkali phase and further comprising passing at least a portion of the hydrocarbon phase through another coalescing zone for obtaining a processed hydrocarbon stream.

4. The process according to claim 1, wherein the hydrocarbon stream comprises one or more C4⁻ hydrocarbons.

5. The process according to claim 3, wherein the processed hydrocarbon stream further comprises no more than about 1 ppm, by weight, of sodium ions.

6. The process according to claim 3, further comprising the hydrocarbon phase rising to a settling zone downstream from the extraction zone.

7. The process according to claim 1, wherein the oleophilic media comprises at least one of a metal mesh that is optionally coated, one or more glass fibers, sand, or anthracite coal.

8. The process according to claim 7, wherein the oleophilic media comprises a metal mesh with a coating.

9. The process according to claim 8, wherein the coating comprises at least one of a fluoropolymer and polypropylene.

10. The process according to claim 3, wherein the another coalescing zone comprises a hydrophilic media.

11. The process according to claim 10, wherein the hydrophilic media comprises at least one of a metal mesh that is optionally coated, fiberglass, or stainless steel mesh.

12. The process according to claim 11, wherein the hydrophilic media comprises a mesh.

13. The process according to claim 12, wherein the mesh comprises a metal mesh with a hydrophilic coating.

14. The process according to claim 12, wherein the mesh comprises a fiberglass.

15. A process for removing one or more sulfur compounds from one or more hydrocarbons, comprising:
A) passing a combined stream comprising one or more hydrocarbons and an alkali to a prewash zone;
B) obtaining from the prewash zone a hydrocarbon stream and passing the hydrocarbon stream into an extraction zone including a first coalescing zone;
C) mixing the hydrocarbon stream with an alkali stream to obtain a hydrocarbon phase and an alkali phase; and
D) passing at least a portion of the hydrocarbon phase to a settling zone containing a second coalescing zone to obtain a processed hydrocarbon stream.

16. The process according to claim 15, wherein all the zones are contained within a single vessel.

17. The process according to claim 15, wherein a hydrocarbon stream upstream of the prewash zone is at a temperature of about 30-about 50° C., and a pressure of about 400-about 1,900 KPa.

18. A vessel for removing one or more sulfur compounds from one or more hydrocarbons, comprising:
A) a prewash zone;
B) an extraction zone downstream of the prewash zone containing a first coalescing zone; and
C) a settling zone downstream of the extraction zone containing a second coalescing zone.

19. The vessel according to claim 18, wherein the first coalescing zone comprises an oleophilic coated mesh.

20. The vessel according to claim 18, wherein the second coalescing zone comprises a hydrophilic coated mesh.
